(19) United States
(12) Patent Application Publication
(10) Pub. No.: US 2004/0034248 A1
(43) Pub. Date: Feb. 19, 2004

(54) Process for preparing gabapentin

(75) Inventors: Sorin Bercovici, Kiriat Ono (IL); Sabar Sasson, Rehovot (IL); Ulanenko Konstantin, Natania (IL)

Correspondence Address: DARBY & DARBY P.C., Post Office Box 5257, New York, NY, 10150-5257, US

(73) Assignees: Taro Pharmaceutical Industries, Ltd.; Taro Pharmaceuticals Ltd.

(21) Appl. No.: 10/414,823

(22) Filed: Apr. 16, 2003

Related U.S. Application Data
(60) Provisional application No. 60/373,412, filed on Apr. 16, 2002.

(51) Int. Cl. C07C 229/32

(52) U.S. Cl. 562/507

(57) ABSTRACT

The present invention provides compositions and methods of preparing gabapentin that includes (a) subjecting cyclohexanediacetic acid monoamide to a Hofmann rearrangement to yield a solution comprising an isocyanate intermediate; (b) hydrolyzing the isocyanate intermediate in the presence of an alkali base to form a gabapentin alkali salt; (c) converting the gabapentin alkali salt to a gabapentin-amine salt in a water-miscible polar solvent; (d) adding a basic or weakly basic ion exchange resin to a solution comprising the gabapentin-amine salt; (e) removing the ion exchange resin from the solution; and (f) concentrating the solution to yield gabapentin.

This application claims the benefit of U.S. Provisional Application No. 60/373,412, filed Apr. 16, 2002, which is hereby incorporated by reference in its entirety.

### FIELD OF THE INVENTION

The present invention relates to a commercially feasible, large-scale, novel process for the preparation of high purity gabapentin, a medicament which is useful in treating certain cerebral disorders.

### BACKGROUND OF THE INVENTION

Gabapentin (GBP), or 1-aminomethyl-1-cyclohexaneacetic acid, is a γ-amino acid having the following chemical structure:

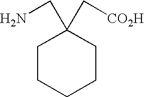

GBP is used to treat certain forms of epilepsy, faintness attacks, hypokinesis, certain cranial traumas, and to improve cerebral function. GBP has a structural relationship to γ-aminobutyric acid (GABA). However, unlike GABA, GBP is capable of crossing the blood-brain cell barrier, and is less toxic in humans than GABA. Various uses of GBP are disclosed in U.S. Pat. Nos. 4,024,175, 4,087,544, and German counterpart Patent Nos. 2460891 and 2543821.

Many methods exist for the preparation of various substituted and unsubstituted cyclic gamma (γ)-amino acids, such as GBP and its derivatives. For example, U.S. Pat. Nos. 4,024,175 and 4,087,544 disclose the preparation of various amine salts, sodium salts and esters of GBP. The procedures described are multistep syntheses based on functionalization of 1,1-cyclohexanediacetic acid. The functionalized diacetic acid derivatives are converted to isocyanate or urethane intermediates by known methods (e.g., Lossen, Curtius or Hofmann rearrangements, see, March, Advanced Organic Chemistry, 3d Ed., Wiley & Sons (1985), pp. 983-85).

The preparation and use of GBP, including certain salts (such as sulfonate salts) and ester derivatives of GBP are also described in German Patent Nos. 2460891 and 2543821 (U.S. Pat. Nos. 4,024,175 and 4,087,544).

U.S. Pat. Nos. 4,894,476 and 4,960,931 describe a large-scale method for converting the sulfate, methanesulfonate, hydrochloride and hydrobromide salts of GBP into the pure, crystalline amino acid monohydrate or anhydrous forms. This method comprises subjecting the salt solution (in deionized water) to chromatographic separation using a basic ion exchange resin, eluting with deionized water, concentrating the eluate by distillation, adding 2-propanol to the wet slurry, cooling, and filtering the solids to give GBP monohydrate. Anhydrous GBP may be obtained by recrystallization of the monohydrate in dry methanol.

Several methods for preparing GBP by a multi-step synthesis based on cyclohexanone functionalization have also been described. These include the preparation of a nitrile, dicarboxylic acid and/or nitrocarboxylic acid intermediates of GBP, which are then subjected to catalytic hydrogenation to yield the free amino acid, often without the need to prepare the amine salt intermediate of GBP. Such methods are described in U.S. Pat. Nos. 5,068,413 and 5,091,567; 5,095,148; 5,132,451; European Patent Publication Nos. 0414275A2; and Helv. Chim. Acta (1999), 73: 309-14.

An improved process for the preparation of cyclic amino acids is disclosed in U.S. Pat. No. 5,319,135. The process described is based on the rationale described above with respect to cyclohexanone functionalization and uses the dinitrile derivative as a starting material. The dinitrile derivative is hydrolyzed to the corresponding cyanoacetic acid ester or alkali salt, which is then catalytically hydrogenated in neutral, basic or acidic media to yield the desired product, often via the corresponding lactam intermediate.

International Publication No. WO 98/28255 describes a method of converting GBP hydrochloride, free of inorganic salts, to the corresponding free amino acid in a composition that includes polymorphic form II of GBP. The method consists of treating a solution of GBP hydrochloride in a suitable solvent with an organic amine and isolating the precipitated amino acid as polymorphic form III. Form III is then converted to form II.

U.S. Pat. No. 6,054,482 discloses a method for preparation of cyclic amino acids substantially free of cyclic lactam. An amino acid salt (e.g., of hydrochloride) is converted to the corresponding free amino acid containing less than 0.5% of the GBP lactam. This is achieved by methods known in the art, such as those described in U.S. Pat. No. 4,024,175 or German Patent Nos. 2460891 and 2543821, discussed above. For example, an amino acid salt intermediate (i.e., GBP hydrochloride or sulfate) is converted to the free amino acid by passing an aqueous solution of the intermediate through a column filled with basic ion-exchange resin, concentrating the eluate, and purifying the crude product by recrystallization from methanol. The patent also describes the conversion of the GBP lactam to the corresponding amine salt by acid hydrolysis. In addition, the patent specifies that the amount of the remaining anion of the mineral acid cannot exceed 20 ppm, thus insuring the long-term stability of the final product and of the respective pharmaceutical compositions.

While there are a variety of methods for preparing and purifying GBP known in the art, these methods have many disadvantages. For example, the conversion of 1,1-cyclohexanediacetic acid into GBP via the Curtius or Lossen rearrangements are industrially impractical due to the inherent production of dangerous intermediates (explosive azides) or expensive, laborious work-up (Lossen). Further, all processes based on 1,1-cyclohexanediacetic acid derivatives involve the preparation of the corresponding intermediates (amino acid salts and crude products) as aqueous solutions, requiring large volumes of liquid, which is impractical for an industrial synthesis. Moreover, the ion exchange step used in these processes requires large amounts of demineralized water, dedicated, large chromatographic columns filled with ion exchange resin, using specialized and costly equipment (i.e., dozatory, pressure pumps and pipes, addition and receiving vessels, etc.), which is time-consuming and also commercially impractical. The isolation of crude amino acids from aqueous solutions by evaporation at temperatures below 40-50° C. requires dedicated equipment, a high energy, time consuming and impractical operation. Alternative methods that do not proceed via the amino acid salts (such as hydrochlorides or sulfates) include multistep laborious syntheses and catalytic hydrogenations that require special, dedicated equipment. Such methods usually involve the presence of the lactam intermediate, and its conversion to the desired, free amino acid is not immediate in neutral conditions and often remains as an undesired impurity in the final product. Moreover, known methods for lactam removal (such as amino acid hydrolysis as described in U.S. Pat. No. 6,054,482) result in the corresponding amino acid hydrochloride or sulfate salts solutions which still suffer from the disadvantages outlined above (e.g., the need for purification via large scale ion exchange chromatographic columns).

Therefore, a need exists for an economical, facile industrial procedure to prepare highly pure, pharmaceutical grade gabapentin and related amino acids.

### SUMMARY OF THE INVENTION

The present invention provides a novel method of preparing gabapentin from a gabapentin-amine salt. The method includes the steps of:

(a) forming a solution comprising the gabapentin-amine salt and an basic ion exchange resin;

(b) removing the ion exchange resin from the suspension to yield a solution comprising gabapentin; and

(c) concentrating the solution formed in step (b) to yield gabapentin.

The invention also provides a method of preparing gabapentin from a gabapentin-amine salt, the method comprising the steps of:

(a) adding a basic or weakly basic ion exchange resin to a solution comprising the gabapentin-amine salt and a water-miscible polar solvent;

(b) filtering or decanting the solution to remove the ion exchange resin from the solution; and

(c) concentrating the solution to yield gabapentin.

Also provided is a method of preparing gabapentin from a gabapentin alkali salt by:

(a) adding a mineral acid to the gabapentin alkali salt in a water-miscible polar solvent to yield a solution of a gabapentin-amine salt;

(b) filtering or decanting the solution,

(c) adding a basic or weakly basic ion exchange resin directly to the filtered or decanted solution;

(d) removing the ion exchange resin from the solution; and

(e) concentrating the solution to provide gabapentin

The invention also provides a method of preparing gabapentin by:

(a) subjecting cyclohexanediacetic acid monoamide to a Hofmann rearrangement to yield a solution comprising an isocyanate intermediate:

(b) hydrolyzing the isocyanate intermediate in the presence of an alkali base to form a gabapentin alkali salt;

(c) converting the gabapentin alkali salt to a gabapentin-amine salt in a water-miscible polar solvent;

(d) adding a basic or weakly basic ion exchange resin to a solution comprising the gabapentin-amine salt;

(e) removing the ion exchange resin from the solution; and

(f) concentrating the solution to yield gabapentin.

Gabapentin having a purity of at least about 98.5% (pharmaceutical-grade) can be prepared by the methods of present invention.

The present invention also provides for a pharmaceutical composition comprising gabapentin initially containing less than 0.5% by weight of a corresponding lactam with respect to the weight of gabapentin and having greater than 20 ppm of an anion of a mineral acid with respect to the weight of gabapentin. In a preferred embodiment, after one year of storage at 25° C. and 60% humidity the conversion of gabapentin to its corresponding lactam does not exceed 0.2% by weight of gabapentin. The composition may further comprise at least one adjuvant. In a preferred embodiment, the adjuvant is selected from modified maize starch, glycerol behenic acid ester, sodium croscarmelose, methacrylic acid co-polymers (types A and C), anion exchangers, titanium dioxide, silica gels hydroxypropylmethylcellulose, polyvinylpyrrolidone, crospovidon, sodium starch glycolate, copolyvidone, maize starch, cyclodextrin, lactose, talc, co-polymers of dimethylamino-methacrylic acid and neutral methacrylic acid ester. The anion of a mineral acid may be a halide. In a preferred embodiment, the amount of the anion of a mineral acid does not exceed 100 ppm.

Another embodiment is gabapentin which contains less than 0.5% of a corresponding lactam with respect to the weight of gabapentin and between 20 and 100 ppm of an anion of a mineral acid (e.g., chloride) with respect to the weight of gabapentin. In a preferred embodiment, after one year of storage at 25° C. and 50% humidity the conversion of gabapentin to the corresponding lactam does not exceed 0.2% by weight of gabapentin.

Yet another embodiment is a pharmaceutical composition comprising gabapentin and at least one adjuvant, and initially containing less than 0.5% by weight of a corresponding lactam with respect to the weight of gabapentin and having greater than 20 ppm of chloride with respect to the weight of gabapentin. In a preferred embodiment, after one year of storage at 25° C. and 50% humidity the conversion of gabapentin to the corresponding lactam does not exceed 0.2% by weight of gabapentin.

The present invention also provides for a pharmaceutical composition comprising gabapentin initially containing less than 0.5% by weight of a corresponding lactam and having pH in the range of 6.8 to 7.3. In a preferred embodiment, after one year of storage at 25° C. and 60% humidity the conversion of gabapentin to its corresponding lactam does not exceed 0.2% by weight of gabapentin. The pharmaceutical composition preferably has a pH in the range of 7.0 to 7.2. The pharmaceutical composition may include one or more adjuvants, such as modified maize starch, sodium croscarmelose, glycerol behenic acid ester, methacrylic acid co-polymers (types A and C), anion exchangers, titanium dioxide, silica gels such as Aerosil 200, hydroxypropylmethylcellulose, polyvinylpyrrolidone, crospovidon, poloxamer 407, poloxamer 188, sodium starch glycolate, copolyvidone, maize starch, cyclodexterin, lactose, talc, co-polymers of dimethylamino-methacrylic acid and neutral methacrylic acid ester.

Yet another embodiment is gabapentin which contains less than 0.5% of the corresponding lactam, and less than 100 ppm of the anion of a mineral acid, which has a pH between 6.8 and 7.3. In a preferred embodiment, after one year at 25° C. and 60% relative humidity, the conversion of gabapentin to its corresponding lactam does not exceed 0.2% by weight of gabapentin.

### BRIEF DESCRIPTION OF THE FIGURES

FIG. 1 is a schematic representation of the preparation of gabapentin by a method of the present invention.

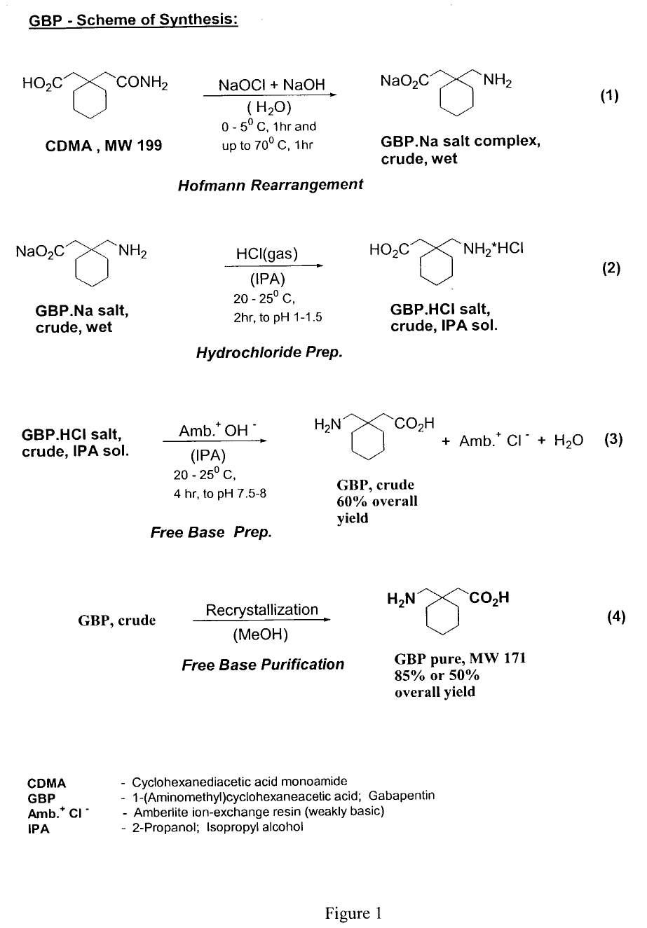

### DETAILED DESCRIPTION OF THE INVENTION

Definitions

The term “CDMA” refers to cyclohexanediacetic acid monoamide which has the formula

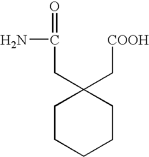

CDMA is available from Cipla Ltd of Mumbai, India, and Interorgana Chemiehandel GmbH & Co. of Hamburg, Germany.

The term “gabapentin” (also herein referred to as GBP) as used herein refers to the free acid of gabapentin having the formula

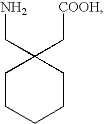

The term “form II” of gabapentin refers to the polymorphic form of gabapentin denoted as form II in International Publication No. WO 98/28255 and U.S. Pat. No. 6,255,526, both of which are hereby incorporated by reference.

The term “gabapentin alkali salt” refers to a salt of gabapentin of the formula:

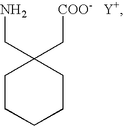

where Y⁺ is an alkali metal, such as sodium (also referred to as gabapentin sodium salt).

The term “gabapentin-amine salt” refers to a salt of gabapentin having the formula:

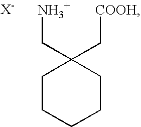

where X⁻ is an anion, such as, for example, chloride. For example, the gabapentin-amine salt may be gabapentin hydrochloride:

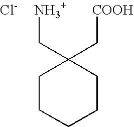

Hofmann Rearrangement of CDMA and Hydrolysis of the Resulting Isocyanate Intermediate

The first step of the method of the present invention is to subject CDMA to a Hofmann rearrangement to yield an isocyanate intermediate. A “Hofmann rearrangement” is a reaction in which a compound of the formula R—C(O)—NH₂ is converted into a compound of the formula R—N═C═O, i.e., an isocyanate. The Hofmann rearrangement may be performed with a hypohalite, such as a hypochlorite or hypobromite. For example, CDMA may be reacted with sodium hydroxide and sodium hypochlorite (or sodium hypobromite) to yield an isocyanate intermediate of the formula

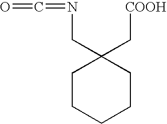

Suitable methods for performing Hofmann rearrangement reactions are known in the art. See, for example, March, Advanced Organic Chemistry, 3d Ed., Wiley & Sons (1985), pp. 983-85; Morrison and Boyd, Organic Chemistry, 5^{th} Ed., Allyn and Bacon, Inc. (1987), p. 1102-1107; and U.S. Pat. No. 4,087,544, which are hereby incorporated by reference. According to a preferred embodiment, the Hofmann rearrangement is performed in an alkaline aqueous solution (e.g., an aqueous sodium hydroxide solution) at a temperature of −10 to 15° C.

A preferred method of performing the Hofmann rearrangement is as follows. CDMA is slowly added to an aqueous solution of sodium hydroxide, while the temperature of the aqueous solution is maintained below 30° C. Preferably, the temperature of the aqueous solution is maintained at −10 to 25° C., and more preferably between 5 and 20° C. A separate vessel is charged with a sodium hypochlorite solution and cooled below 5° C. Sodium hydroxide is then added to the hypochlorite solution, while maintaining the temperature of the hypochlorite solution below 10° C. The hypochlorite solution is then cooled below 0° C., and the CDMA solution is added to the hypochlorite solution, while the temperature of the hypochlorite solution is maintained at 0-20° C. The conversion of CDMA to the isocyanate intermediate typically is near or at completion after several hours.

The isocyanate intermediate is hydrolyzed in situ in the presence of an alkali base (such as sodium hydroxide) by any method known in the art to yield gabapentin alkali salt, such as gabapentin sodium salt. Preferably, the isocyanate intermediate is hydrolyzed by basic hydrolysis with aqueous sodium hydroxide to form gabapentin sodium salt. When the Hofmann rearrangement is performed in an aqueous solution containing sodium hydroxide, the isocyanate formed in the solution hydrolyzes to yield gabapentin sodium salt. The hydrolysis is preferably performed with fast heating.

Preferably, any excess hypochlorite reagent present is decomposed in situ by addition of a reducing agent, such as sodium sulfide, sodium hypophosphite or sodium thiosulfate. Preferably, the excess hypochlorite reagent is decomposed at a temperature of 5-20° C. In one embodiment, a reducing agent is added until no hypochlorite reagent is detectable in the solution. The presence of hypochlorite can be determined by any method known in the art, such as with a potassium iodide-starch paper test. In a preferred embodiment, a saturated sodium thiosulfate pentahydrate solution is added at 5-20° C. until the solution tests negative in a potassium iodide-starch paper test. The mixture is then heated and cooled to room temperature.

The gabapentin alkali salt formed may be isolated by any method known in the art. For example, the gabapentin alkali salt may be isolated as a wet solid from the aqueous solution by filtering or decanting. In one embodiment of the present invention, a salting-out procedure is used to precipitate the gabapentin alkali salt. For example, an excess (e.g., about 2 to about 4 molar excess) of alkali base (e.g., sodium hydroxide) may be added to the solution to salt out the gabapentin alkali salt. The wet gabapentin alkali salt obtained may optionally be washed with alcohol, such as isopropanol.

Conversion of Gabapentin Alkali Salt to Gabapentin-Amine Salt

The gabapentin alkali salt is converted to a gabapentin-amine salt by any method known in the art. Preferably, the gabapentin-amine salt is gabapentin hydrochloride or gabapentin hydrogen sulfate, with gabapentin hydrochloride being most preferred.

The gabapentin alkali salt may be converted to a gabapentin-amine salt by reacting the gabapentin alkali salt with a mineral acid. Preferably, the gabapentin alkali salt is first combined with a water-miscible, polar solvent and then reacted with the mineral acid.

The gabapentin alkali salt (such as gabapentin sodium salt) may be wet, i.e., it may contain water. Therefore, the wet gabapentin alkali salt obtained in the prior step (from hydrolysis of the isocyanate intermediate) may be directly converted to a gabapentin-amine salt without first drying it. According to one embodiment, after combining the gabapentin alkali salt with the water-miscible, polar solvent, the solvent comprises from about 5% to about 18% water by volume, and more preferably from about 8 to about 14% water.

The mineral acid is preferably a strong acid. Suitable mineral acids include, but are not limited to, hydrochloric acid, hydrobromic acid, nitric acid, phosphoric acid, and sulfuric acid. Preferred mineral acids include, but are not limited to, sulfuric acid and hydrochloric acid.

The water miscible, polar solvent may be a C₁-C₆ alkyl alcohol, ketone, or ether. Suitable C₁-C₆ alkyl alcohols include, but are not limited to, methanol, ethanol, 2-propanol (isopropanol), butanol (such as n-butanol, isobutanol, and t-butanol), and isoamyl (isopentanol) alcohol. Preferred C₁-C₆ alkyl alcohols include, but are not limited to, ethanol and 2-propanol. Suitable ketones include, but are not limited to, acetone and butanone. Suitable ethers include, but are not limited to, diisopropyl ether, t-butylmethylether, tetrahydrofuran, and dimethoxyethane.

The insoluble inorganic salts formed during the conversion may be removed by filtration or other methods known in the art. The gabapentin-amine salt solution may be used as is in the next step.

Converting the Gabapentin-Amine Salt to Gabapentin with an Ion Exchange Resin

The gabapentin-amine salt in solution is converted to gabapentin by the direct addition of an ion exchange resin to the reaction medium as a solid phase reagent. Any suitable reaction vessel (e.g., glass flask, glass-lined or stainless steel reactor) can be used instead of a classical, static solid phase ion exchange column. Preferably, the solution containing the ion exchange resin is stirred at a temperature between about 10 and about 40° C. and more preferably between about 20 and about 35° C. (e.g., room temperature).

Generally, the ion exchange reaction is complete when the pH of the solution is slightly basic (i.e., a pH of at least about 7), preferably between about 7 and about 9. The exhausted ion exchange resin may be removed from the solution by any method known in the art, such as decantation or filtration. Free of solids, the solution may be used directly in the next step.

Any suitable ion exchange resin may be used. The ion exchange resin is preferably in pearl or granular form. Preferably, the ion exchange resin is basic or weakly basic, with weakly basic resins being most preferred. Non-limiting examples of suitable ion exchange resins include Amberlite™ IRA-67 (Rohm and Haas, Philadelphia, Pa.), Amberlite™ IRA-93 (Rohm and Haas), Lewatit™ MP-62 (Sybron Chemicals, Birmingham, N.J.), Amberlyst A21 (Rohm & Haas), and Dowex™ AMW 500 (Dow Chemical, Midland, Mich.).

Concentrating the Gabapentin Containing Solution

The gabapentin containing solution from the prior step may be concentrated by any method known in the art and, optionally, purified to obtain gabapentin. Preferably, anhydrous gabapentin is formed. The gabapentin-containing solution may be concentrated by, for example, azeotropic distillation. Preferably, the azeotropic distillation is performed at a temperature of about 25 to about 60° C. and more preferably at about 35 to about 50° C. The azeotropic distillation is preferably performed at a pressure of about 30 to about 300 mm Hg and more preferably a pressure of about 50 to about 100 mm Hg.

The gabapentin obtained may be further purified by methods known in the art, such as recrystallization or slurrying the gabapentin in a suitable solvent (e.g., a C₁-C₆ alkyl alcohol such as those described above). Preferably, the gabapentin is recrystallized in a suitable solvent, such as a C₁-C₆ lower alkyl alcohol. Preferred C₁-C₆ lower alkyl alcohols include, but are not limited to, methanol, ethanol, 2-propanol (IPA), and mixtures thereof.

For example, the gabapentin can be recrystallized by dissolving it in a hot organic solvent, cooling, filtering, and drying. Preferred solvents include ethanol and isopropanol. The solution of gabapentin and organic solvent may be stirred to aid dissolution. Preferably the organic solvent is maintained at about 25 to about 80° C. during dissolution and more preferably at about 35 to about 65° C. Before filtering, the solution is typically cooled below about 10° C., and preferably cooled to about −5 to about 5° C.

The method of the present invention can produce gabapentin having a purity of at least about 90%, more preferably at least about 95%, and most preferably at least about 98.5%, i.e., pharmaceutical grade gabapentin (98.5-100% pure).

### EXAMPLES

The present invention is described by the following non-limiting Examples.

### Example 1

Step 1. Preparation of Crude Gabapentin Sodium (GBP-Na) Salt:

A fresh solution of 1,1-cyclohexanediacetic acid monoamide (CDMA) was prepared by slow addition of monoamide (CDMA, 0.5 mole, 100 g) into a solution (120 g, 107 ml) containing about 15% of sodium hydroxide in water, while the temperature was kept below 15° C. At the same time, a suitable vessel was charged with a sodium hypochlorite 8-10% solution (485 g) and cooled below 5° C. Sodium hydroxide pellets (100 g) were then added in small portions, while the temperature was kept below 10° C. The solution was then cooled below 0° C.

The thick syrupy CDMA solution was then added to the cooled hypochlorite solution with stirring, while the temperature was maintained at 0-10° C. After 2-3 hours of stirring at the same temperature, a saturated sodium thiosulfate pentahydrate solution was added to the cold mixture until negative to a potassium iodide-starch paper test. The reaction mixture was then heated to 60-70° C. for about ½ hour and cooled to room temperature.

The white solid precipitate was filtered in vacuo and washed with a small amount of isopropyl alcohol.

Yield: 203 g of crude, wet GBP-Na salt or 131 g on dry basis containing about 63 g of sodium salt (65% yield, by HPLC analysis vs. gabapentin standard). The crude, wet salt is directly used in the next step.

Step 2. Preparation of Gabapentin Hydrochloride Salt Solution:

Crude wet GBP sodium salt (203 g) was suspended in 2-propanol (IPA, 600 mL). A slow, constant flow of hydrogen chloride was bubbled into the well-stirred suspension at room temperature, until the pH became acidic, i.e., pH 1-5.

The inorganic salts that resulted were removed by filtration. The clear filtrate containing the solution of GBP hydrochloride was directly used in the next step.

Step 3. Preparation and Isolation of Gabapentin:

Amberlite™ IRA-68 basic ion exchange resin (Rohm and Haas, Philadelphia, Pa., 200 g) was added in two portions to the GBP hydrochloride solution obtained in step 2. The mixture was stirred at room temperature for 4-6 hours. When the exchange was completed (pH 7-9), the resin was separated and the clear solution was then concentrated to about ⅓ of the initial volume by azeotropic distillation at reduced pressure (20-45° C. and 10-150 mm Hg). Fresh, dry isopropanol (300-400 mL) was added to the residue and the mixture was distilled again.

The resultant suspension was cooled (0-5° C.) and the white solid isolated by suction filtration was dried overnight in a vacuum oven to yield GBP white crystals.

| | | |
|---|---|---|
| Yield: | 50 g (90% from the sodium salt and 59% | |
| | overall yield) | |
| Identification: | HPLC vs. standard | |
| Melting Range: | 159-160.6° C. | |
| Purity (HPLC): | gabapentin content: | 98.1% |
| | related lactam: | 0.1% |
| | unknown impurities: | 1.0% |
| Water content | 0.5% | |
| (K. Fisher titration): | | |

Step 4. Preparation of Pure Gabapentin:

GBP (50 g, obtained in step 3) was added to hot methanol (500 ml; 50-60° C. The mixture was heated to reflux until fully dissolved. If the solution remained turbid it was filtered hot and then concentrated by low pressure distillation (20-40° C. and 10-150 mmHg) to about ½ of the initial volume (about 230 mL of the methanol was removed). The resultant suspension was gradually cooled to 0-5° C., with stirring. After 2-4 hours, the white solid was isolated by filtration, washed with cold methanol and dried in a vacuum oven, to yield pure GBP as bright white crystals.

| Yield: | 40 g (80% or 47% overall yield based on | |
|---|---|---|
| | CDMA). | |
| Identification: | (FT-IR, vs. authentic commercial sample): | |
| | identical | |
| Assay: | 100% (HPLC vs. standard). | |
| Purity (HPLC): | gabapentin content: | 99.6% |
| | lactam: | 0.08% |
| | related diacetic acid (CDAA): | 0.2% |
| | unknown impurities: | 0.1% |
| | chloride content: | 88 ppm |
| Water (KF): | 0.2% | |

### Example 2

Step 1. Preparation of Crude Gabapentin Sodium Salt:

Crude GBP sodium salt was prepared according to the method outlined in step 1 of Example 1, above, except the alkaline monoamide solution (from 0.5 mole, 100 g CDMA) was added at 0° C. to a sodium hypobromite solution prepared in situ by dissolution of bromine (0.6 mole, 96 g) in cold (0° C.) sodium hydroxide solution (20%, 220 ml). Additional isolation and purification was performed as described in Example 1, to yield a white solid precipitate.

Yield: 271 g of crude, wet GBP-Na salt or 152 g on dry basis containing about 65 g of sodium salt (67% yield, by HPLC analysis).

Steps 2 and 3:

The crude, wet sodium salt isolated by the method outlined above in step 1 (of Example 2) was converted into GBP according to the method described in Steps 2 and 3 of Example 1, with the following results:

| | | |
|---|---|---|
| Yield: | 23 g (27% overall yield) as white crystals. | |
| Identification: | positive (by FT-IR) | |
| Purity (HPLC): | gabapentin content: | 98.8% |
| | related lactam: | 0.1% |
| | related diacetic acid (DCAA): | 0.5% |
| | unknown impurities: | 0.6% |
| | halide content (Br⁻ and Cl⁻): | 340 ppm |

### Example 3

Step 1 was carried out according to the method outlined in Step 1 of Example 1 using 100 g (0.5 mole) of monoamide.

Step 2. Preparation of Gabapentin Hydrogen Sulfate Salt Solution:

A 1/1 w/w solution of sulfuric acid in 2-propanol (IPA) was prepared by careful addition of the concentrated acid (94-98%; 181 g, 100 mL) to cold IPA (188 g, 250 mL), while the temperature was kept below 15° C. The acid solution was then added to the crude, wet GBP-sodium salt (256 g) suspended in IPA (400 mL) at 20-30° C., until a constant acidic pH value was achieved, i.e., pH 2-5. The insoluble salts were removed by filtration and the clear filtrate containing the GBP-hydrogen sulfate was used directly in the next step.

Steps 3 and 4:

The clear solution of GBP-salt obtained above was worked up as described in Steps 3 and 4 of Example 1 to yield pure GBP as bright-white crystals.

| Yield: | 36 g (42% overall yield) | |
|---|---|---|
| Identification: | (FT-IR fingerprint, vs. authentic sample): | |
| | positive | |
| Purity (HPLC): | gabapentin content: | 99.7% |
| | lactam: | 0.05% |
| | diacetic acid (CDAA): | 0.1% |
| | unknown impurities: | 0.1% |
| | chloride content: | 26 ppm |
| | sulfate content: | about |
| | | 170 ppm |
| Water (KF): | 0.1% | |

### Example 4

Step 1 was carried out according to the method outlined in Step 1 of Example 1, using 50 g (0.25 mole) monoamide to give 138 g crude wet sodium salt of GBP.

Step 2. Preparation of Gabapentin Hydrogen Chloride Salt:

The crude, wet sodium salt prepared according to the method described above (121 g, 80 g on dry basis) was suspended in ethanol (200 mL). Hydrogen chloride was then bubbled into the stirred mixture at 22-30° C., until an acidic pH value was achieved. The suspension was further stirred for ½ hour and filtered. The clear filtrate containing GBP-salt solution in ethanol was used directly in the next step.

Step 3. Preparation and Isolation of Gabapentin:

This step was performed as described in Example 1, using an ethanolic solution of GBP hydrochloride and Amberlite™ IRA-67 ion exchange resin (110 g) to yield GBP as white crystals, upon azeotropic distillation.

| | | |
|---|---|---|
| Yield: | 17 g (38% overall yield). | |
| Identification: | (FT-IR): positive | |
| Melting Range: | 159.9-161.3° C. | |
| Purity (HPLC): | gabapentin content: | 98.7% |
| | lactam: | 0.1% |
| | diacetic acid (CDAA): | 0.6% |
| | unknown impurities: | 0.5% |
| | chloride content: | about 580 ppm |

### Example 5

Step 1. Preparation of Crude Gabapentin Sodium Salt:

Sodium hydroxide pellets (1.5 kg) were added in small portions into a cold (0° C.) sodium hypochlorite 10% solution, keeping the inside temperature below 15° C. The solution obtained was further cooled to 0-5° C.

In a separate vessel, monoamide (CDMA, 1.5 kg, 7.5 mole) was added to a cold (0-5° C.), dilute solution of sodium hydroxide prepared from sodium hydroxide 50% (d=1.4 g/ml, 0.75 kg) in demineralized water, with stirring and cooling.

The CDMA suspension was slowly added to the alkaline sodium hypochlorite solution keeping the inside temperature below 10° C. The reaction mixture was stirred for 2-3 hours, and then a sodium thiosulfate 50% solution was added until a negative test with a potassium iodide starch paper was achieved. The reaction mixture was then rapidly heated to 60-70° C., stirred for ½ hour and cooled to room temperature.

Filtration of the resulting suspension produced the crude sodium salt intermediate as off-white solid.

Yield: 4.2 kg wet or 2.9 kg on dry basis (LOD 31%).

Gabapentin content (HPLC, %): 36%, which is equivalent to 1.04 kg of sodium salt (72% yield).

Purity: lactam: 0.5% related diacetic acid (CDAA): 0.2%

The crude, wet intermediate was used directly in the next step.

Step 2. Preparation of Gabapentin Hydrochloride Salt:

The crude GBP sodium salt prepared above was suspended in 2-propanol (IPA, 10 kg, 13 L). Hydrogen chloride as a constant, slow stream of gas was bubbled into the stirred suspension, keeping the temperature at room temperature. The bubbling stopped releasing gas when the suspension became acidic, i.e., pH 1-5, and then the insoluble salts were removed by filtration and the clear filtrate (about 14 L) containing GBP hydrochloride solution was used directly in the next step.

Step 3. Preparation and Isolation of Gabapentin:

Amberlite™ IRA-67 ion exchange resin (3.5 kg) was added in two portions to the stirred isopropanol solution (14 L) of GBP hydrochloride obtained above. The suspension was stirred at 15-35° C. for 2-3 hours until a constant pH value of 8-8.5 was reached, and then the resin was removed by filtration and the solution was further filtered through a celite bed. The clear solution of GBP in IPA/water was concentrated to about ⅓ from its initial volume by azeotropic distillation at 30-40° C. (about 8-10 L of IPA/water was removed). The solvent was eventually replaced with fresh, dry IPA (8-10 L) and the solution was re-distilled until a suitable water content was achieved.

The suspension was cooled (0-5° C.), filtered and washed with cold IPA to yield the desired, wet product, which was dried in a vacuum oven for 8-10 hours to yield GBP as white crystals.

| Yield: | 0.8 kg (62% overall yield from | |
|---|---|---|
| | CDMA) | |
| Identification by m.p.: | 159.9-160.5° C. (dec.) | |
| Assay (HPLC vs. standard): | 98.6% | |
| Purity (HPLC vs. standard): | gabapentin: | 99.7% |
| | lactam: | 0.1% |
| | diacetic acid (CDAA): | 0.1% |
| | unknown impurities: | 0.1% |
| Water (KF): | 0.5% | |

Step 4. Preparation of Pure Gabapentin:

GBP (0.8 kg), prepared above was suspended in methanol (4 L). The suspension was stirred at room temperature and then gently heated to 30-40° C. for 1-2 hours, before being gradually cooled to room temperature and then to 0-5° C. The white, crystalline solid was separated by filtration and washed with cold methanol. The solid was dried in a vacuum oven for 8-10 hours to yield the pure GBP as bright-white crystals.

| | | |
|---|---|---|
| Yield: | 0.6 kg (75% from the previous | |
| | step, or 45% overall yield) | |
| Identification: | (FT-IR vs. standard): complies | |
| Assay: | 99.1% | |
| Purity: | gabapentin: | 100% |
| | lactam: | 0.05% |
| | diacetic acid (CDAA): | 0.03% |
| | unknown impurities: | 0.01% |
| | | (LOD) |
| | | (Limit of Detection) |
| Water (KF): | 0.1 | |
| pH (5% sol): | 7.2 | |
| Chlorides: | about 100 ppm | |
| Sulfates: | not detected. | |

The foregoing examples are designed to illustrate rather than limit the scope of the present invention. All publications, patents, and procedures referenced in this application are incorporated herein by reference.

What is claimed is:

1. A method of preparing gabapentin comprising the steps of:
(a) subjecting cyclohexanediacetic acid monoamide to a Hofmann rearrangement to yield a solution comprising an isocyanate intermediate:
(b) hydrolyzing the isocyanate intermediate in the presence of an alkali base to form a gabapentin alkali salt;
(c) converting the gabapentin alkali salt to a gabapentin-amine salt in a water-miscible polar solvent;
(d) adding a basic or weakly basic ion exchange resin to a solution comprising the gabapentin-amine salt;
(e) removing the ion exchange resin from the solution; and
(f) concentrating the solution to yield gabapentin.

2. The method of claim 1, further comprising:
(g) purifying the gabapentin obtained in step (f) by recrystallizing the gabapentin from a C₁-C₆ lower alkyl alcohol, or slurrying the gabapentin in a C₁-C₆ lower alkyl alcohol.

3. The method of claim 1, wherein step (a) further comprises adding a reducing agent to the solution containing the isocyanate intermediate.

4. The method of claim 3, wherein the reducing agent is added until the solution tests negative in a potassium iodide-starch paper test.

5. The method of claim 4, wherein the reducing agent is sodium thiosulfate pentahydrate.

6. The method of claim 1, wherein step (b) further comprises isolating the gabapentin alkali salt.

7. The method of claim 6, wherein the gabapentin alkali salt is isolated by filtration or decantation.

8. The method of claim 7, wherein the gabapentin alkali salt is gabapentin sodium salt.

9. The method of claim 7, wherein the water-miscible polar solvent in step (c) comprises a C₁-C₆ lower alkyl alcohol.

10. The method of claim 8, wherein the gabapentin sodium salt isolated in step (b) is wet.

11. The method of claim 10, wherein the water-miscible polar solvent in step (c) comprises from about 5% to about 18% water by volume after combining the wet gabapentin sodium salt therewith.

12. The method of claim 11, wherein step (c) further comprises filtering the reaction medium to provide a filtrate containing the gabapentin-amine salt.

13. The method of claim 12, wherein the gabapentin-amine salt is gabapentin hydrochloride or gabapentin hydrogen sulfate.

14. The method of claim 12, wherein the solution in step (d) comprises the filtrate from step (c).

15. The method of claim 14, wherein the gabapentin-amine salt is gabapentin hydrochloride or gabapentin hydrogen sulfate.

16. The method of claim 1, wherein the ion exchange resin is a weakly basic ion exchange resin.

17. The method of claim 1, wherein the ion exchange resin is in pearl or granular form.

18. The method of claim 1, wherein the gabapentin yielded in step (f) is at least about 98.5% pure.

19. A method of preparing gabapentin from a gabapentin alkali salt comprising the steps of:
(a) adding a mineral acid to the gabapentin alkali salt in a water-miscible polar solvent to yield a solution of a gabapentin-amine salt;
(b) filtering or decanting the solution,
(c) adding a basic or weakly basic ion exchange resin directly to the filtered or decanted solution;
(d) removing the ion exchange resin from the solution; and
(e) concentrating the solution to provide gabapentin

20. The method of claim 19, wherein the solution in step (a) comprises from about 5 to about 18% water by volume.

21. The method of claim 19, wherein the ion exchange resin is a weakly basic ion exchange resin.

22. The method of claim 19, wherein the ion exchange resin is in pearl or granular form.

23. The method of claim 19, wherein the gabapentin alkali salt is gabapentin sodium salt.

24. The method of claim 19, wherein the mineral acid is hydrochloric acid or sulfuric acid.

25. A method of preparing gabapentin from a gabapentin-amine salt, the method comprising the steps of:
(d) adding a basic or weakly basic ion exchange resin to a solution comprising the gabapentin-amine salt and a water-miscible polar solvent;
(e) filtering or decanting the solution to remove the ion exchange resin from the solution; and
(f) concentrating the solution to yield gabapentin.

26. The method of claim 25, wherein the ion exchange resin is a weakly basic ion exchange resin.

27. The method of claim 25, wherein the ion exchange resin is in pearl or granular form.

28. The method of claim 25, wherein the gabapentin-amine salt is gabapentin hydrochloride or gabapentin hydrogen sulfate.

29. The method of claim 25, wherein the solution comprises a C₁-C₆ lower alkyl alcohol.

30. The method of claim 29, wherein the solution comprises from about 5 to about 18% water by volume.

31. The method of claim 30, wherein the gabapentin-amine salt is gabapentin hydrochloride or gabapentin hydrogen sulfate.

32. The method of claim 26, wherein step (b) is performed when the pH of the solution is at least about 7.

33. The method of claim 25, wherein step (a) comprises stirring the solution at room temperature until the pH of the solution is at least about 7.

34. A pharmaceutical composition comprising gabapentin and initially containing less than 0.5% by weight of a corresponding lactam with respect to the weight of gabapentin and having greater than 20 ppm of an anion of a mineral acid with respect to the weight of gabapentin, which, after one year of storage at 25° C. and 60% humidity the conversion of gabapentin to its corresponding lactam does not exceed 0.2% by weight of gabapentin.

35. The pharmaceutical composition of claim 34 further comprising at least one adjuvant.

36. The pharmaceutical composition of claim 35, wherein at least one adjuvant is selected from the group consisting of modified maize starch, glycerol behenic acid ester, sodium croscarmelose, methacrylic acid co-polymers (types A and C), anion exchangers, titanium dioxide, silica gels hydroxypropylmethylcellulose, polyvinylpyrrolidone, crospovidon, sodium starch glycolate, copolyvidone, maize starch, cyclodextrin, lactose, talc, co-polymers of dimethylamino-methacrylic acid and neutral methacrylic acid ester.

37. The pharmaceutical composition of claim 34, wherein said anion of a mineral acid is a halide.

38. The pharmaceutical composition of claim 34, wherein the amount of said anion of a mineral acid does not exceed 100 ppm.

39. Gabapentin which contains less than 0.5% of a corresponding lactam with respect to the weight of gabapentin and between 20 and 100 ppm of an anion of a mineral acid with respect to the weight of gabapentin, and which, after one year of storage at 25° C. and 50% humidity the conversion of gabapentin to the corresponding lactam does not exceed 0.2% by weight of gabapentin.

40. A pharmaceutical composition comprising gabapentin and at least one adjuvant, and initially containing less than 0.5% by weight of a corresponding lactam with respect to the weight of gabapentin and having greater than 20 ppm of chloride with respect to the weight of gabapentin, which, after one year of storage at 25° C. and 50% humidity the conversion of gabapentin to the corresponding lactam does not exceed 0.2% by weight of gabapentin.

41. A pharmaceutical composition comprising gabapentin and initially containing less than 0.5% by weight of a corresponding lactam with respect to the weight of gabapentin and having greater than 20 ppm of an anion of a mineral acid with respect to the weight of gabapentin.

42. The pharmaceutical composition of claim 41 further comprising at least one adjuvant.

43. The pharmaceutical composition of claim 42, wherein at least one adjuvant is selected from the group consisting of modified maize starch, glycerol behenic acid ester, sodium croscarmelose, methacrylic acid co-polymers (types A and C), anion exchangers, titanium dioxide, silica gels hydroxypropylmethylcellulose, polyvinylpyrrolidone, crospovidon, sodium starch glycolate, copolyvidone, maize starch, cyclodextrin, lactose, talc, co-polymers of dimethylamino-methacrylic acid and neutral methacrylic acid ester.

44. The pharmaceutical composition of claim 41, wherein said anion of a mineral acid is a halide.

45. The pharmaceutical composition of claim 41, wherein the amount of said anion of a mineral acid does not exceed 100 ppm.

46. Gabapentin which contains less than 0.5% of a corresponding lactam with respect to the weight of gabapentin and between 20 and 100 ppm of an anion of a mineral acid with respect to the weight of gabapentin.

47. A pharmaceutical composition comprising gabapentin and at least one adjuvant, and initially containing less than 0.5% by weight of a corresponding lactam with respect to the weight of gabapentin and having greater than 20 ppm of chloride with respect to the weight of gabapentin.

48. A pharmaceutical composition comprising gabapentin initially containing less than 0.5% by weight of a corresponding lactam and having pH in the range of 6.8 to 7.3, which, after one year of storage at 25° C. and 60% humidity the conversion of gabapentin to its corresponding lactam does not exceed 0.2% by weight of gabapentin.

49. The pharmaceutical composition of claim 48, wherein the pH is in the range of 7.0 to 7.2.

50. The pharmaceutical composition of claim 48 further comprising at least one adjuvant.

51. The pharmaceutical composition of claim 50, wherein said adjuvant is selected from the group consisting of modified maize starch, sodium croscarmelose, glycerol behenic acid ester, methacrylic acid co-polymers (types A and C), anion exchangers, titanium dioxide, silica gels such as Aerosil 200, hydroxypropylmethylcellulose, polyvinylpyrrolidone, crospovidon, poloxamer 407, poloxamer 188, sodium starch glycolate, copolyvidone, maize starch, cyclodexterin, lactose, talc, co-polymers of dimethylamino-methacrylic acid and neutral methacrylic acid ester.

52. Gabapentin which contains less than 0.5% of the corresponding lactam, and less than 100 ppm of the anion of a mineral acid, which has a pH between 6.8 and 7.3, and which, after one year at 25° C. and 60% relative humidity, the conversion of gabapentin to its corresponding lactam does not exceed 0.2% by weight of gabapentin.

53. A pharmaceutical composition comprising gabapentin initially containing less than 0.5% by weight of a corresponding lactam and having pH in the range of 6.8 to 7.3.

54. The pharmaceutical composition of claim 53, wherein the pH is in the range of 7.0 to 7.2.

55. The pharmaceutical composition of claim 53 further comprising at least one adjuvant.

56. The pharmaceutical composition of claim 55, wherein said adjuvant is selected from the group consisting of modified maize starch, sodium croscarmelose, glycerol behenic acid ester, methacrylic acid co-polymers (types A and C), anion exchangers, titanium dioxide, silica gels such as Aerosil 200, hydroxypropylmethylcellulose, polyvinylpyrrolidone, crospovidon, poloxamer 407, poloxamer 188, sodium starch glycolate, copolyvidone, maize starch, cyclodexterin, lactose, talc, co-polymers of dimethylamino-methacrylic acid and neutral methacrylic acid ester.

57. Gabapentin which contains less than 0.5% of the corresponding lactam, and less than 100 ppm of the anion of a mineral acid, which has a pH between 6.8 and 7.3.
